(19) United States
(12) Patent Application Publication
(10) Pub. No.: US 2015/0174021 A1
(43) Pub. Date: Jun. 25, 2015

(54) COSMETIC COMPOSITION COMPRISING BENZYLSULFONYL-D-SER-HOMOPHE-(4-AMIDINO-BENZYLAMIDE) AND A POLYALCOHOL

(71) Applicant: DSM IP ASSETS B.V., Heerden (NL)

(72) Inventors: Remo Campiche, Basel (CH); Rainer Voegeli, Basel (CH); Peter Wikstroem, Basel (CH)

(21) Appl. No.: 14/414,004

(22) PCT Filed: Jul. 8, 2013
(86) PCT No.: PCT/IB2013/055564

§ 371 Date: Jan. 9, 2015

(30) Foreign Application Priority Data
Jul. 12, 2012 (EP) 12176040.9

(51) Int. Cl. A61K 8/34; A61Q 19/00; A61K 8/64; A61Q 5/00

(52) U.S. Cl. CPC A61K 8/345; A61Q 5/006; A61Q 19/007; A61K 8/64; A61Q 19/00; A61K 2800/5922

(57) ABSTRACT

The present invention relates to the field of active compositions for use in cosmetic products for humans. More particularly, the present invention relates to a cosmetic composition comprising benzylsulfonyl-D-Ser-homoPhe-(4-amidino-benzylamide), or a salt thereof, and a polyalcohol. Moreover, it relates to the use of said composition for maintaining, restoring, and/or improving the skin hydration, and/or the transepidermal water loss level in human skin. The present invention further relates to the use of benzylsulfonyl-D-Ser-homoPhe-(4-amidino-benzylamide), or a salt thereof, in combination with a polyalcohol for the co-inhibition of both plasmin and urokinase activity in the skin and/or the scalp.

The present invention relates to the field of active compositions for use in cosmetic products for humans. More particularly, the present invention relates to a cosmetic composition comprising benzylsulfonyl-D-Ser-homoPhe-(4-amidino-benzylamide), or a salt thereof, and a polyalcohol. Moreover, it relates to the use of said composition for maintaining, restoring, and/or improving the skin hydration, and/or the transepidermal water loss level in human skin. The present invention further relates to the use of benzylsulfonyl-D-Ser-homoPhe-(4-amidino-benzylamide), or a salt thereof, in combination with a polyalcohol for the co-inhibition of both plasmin and urokinase activity in the skin and/or the scalp.

4-amidino benzylamines such as for example benzylsulfonyl-D-Ser-homoPhe-(4-amidino-benzylamide) have previously been described as urokinase inhibitors (WO2009/026949). Urokinase (uPA), also called urokinase-type plasminogen activator, is a multidomain serine protease (EC 14.21.31). uPA is a 411 amino acid residue protein consisting of three domains: the growth factor-like domain (aa 4-43), the kringle domain (aa 47-135) and the catalytic “B” chain (amino acids 144-411) The kringle domain appears to bind heparin. The growth factor-like domain bears some similarity to the structure of epidermal growth factor (EGF), and is thus referred to as an EGF-like domain. uPA is synthesized as a zymogen (pro-uPA or single chain uPA), and is activated by proteolytic cleavage by plasmin between Lys158 and Ile159. The two resulting chains are kept together by a disulfide bond.

A principal substrate for uPA is plasminogen which is converted by cell surface bound uPA to plasmin. uPA is highly specific to a single peptide linkage in plasminogen. Activated plasmin degrades components of the extracellular matrix (fibrin, fibronectin, laminin, and proteoglycans) and also activates matrix metalloproteases (MMPs) thus promoting the degradation of collagen.

Therefore, it would be beneficial to find a cosmetic composition which not only is a potent selective inhibitor of the urokinase, but is also a potent plasmin inhibitor while not inhibiting serine proteases, including kallikrein 5, kallikrein 7, elastase, factor VII, factor X and tissue-type plasminogen activator (tPA).

Surprisingly, the present inventors found that a cosmetic composition comprising one of the urokinase inhibitors described in WO2009/026949 namely benzylsulfonyl-D-Ser-homoPhe-(4-amidino-benzylamide), and a polyalcohol solves the problems described above, and can be used for topical application for maintaining, restoring, and/or improving the skin hydration, the epidermal barrier, and/or the transepidermal water loss level in human skin. This surprising effect of this cosmetic composition is due to a strong inhibitory effect of the composition against both plasmin and urokinase.

### DESCRIPTION OF THE INVENTION

The present invention therefore relates to a cosmetic composition comprising
a) benzylsulfonyl-D-Ser-homoPhe-(4-amidino-benzylamide), or a salt thereof, and
b) a polyalcohol.

The term “cosmetic composition” as used in the present application refers to cosmetic compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Compositions”, Verlag für chernische Industrie (ed. H. Ziolkowsky), 4^{th} edition, 1992. Preferably, the cosmetic composition according to the present invention is topically applicable.

benzylsulfonyl-D-Ser-homoPhe-(4-amidino-benzylamide) has the following formula (i).

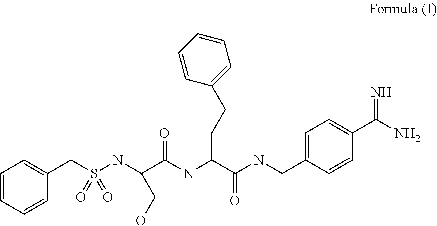 [SMILES: N=C(N)c1ccc(CNC(=O)C(CCc2ccccc2)NC(=O)C(CO)NS(=O)(=O)Cc2ccccc2)cc1]

The compound of formula (I) can be chemically produced by the person skilled in the art according to conventional peptide synthesis chemistry, and also more specifically based on the procedures described in WO 03/076391. This compound can be conveniently used as a racemic mixture but more preferably as pure enantiomer. Preferably, for all the embodiments according to the present invention, the compound of formula (I) is in the form of a cosmetically acceptable salt. Most preferred salt is an acetate salt.

A polyalcohol also known as polyhydric alcohol, or polyol, or sugar alcohol is a hydrogenated form of carbohydrate, whose carbonyl group (aldehyde or ketone, reducing sugar) has been reduced to a primary or secondary hydroxyl group (hence the alcohol). Sugar alcohols have the general formula H(HCHO)ₙ₊₁H, whereas sugars have H(HCHO)ₙHCO. Preferred polyalcohols according to the present invention are selected from the list consisting of: glycol, glycerol, erythritol, threitol, arabitol, xylitol, ribitol, mannitol, sorbitol, galactitol, fucitol, iditol, inositol, maltitol, lactitol, propylenglycol, butylene glycol, hexylene glycol, ethoxydiglycol, dipropylene glycol, and polyglycitol. Most preferred cosmetic composition according to the present invention comprises benzylsulfonyl-D-Ser-homoPhe-(4-amidino-benzylamide), or a salt thereof, and glycerol also named glycerin or 1,2,3-propanetriol.

In a preferred embodiment, the cosmetic composition according to the present invention comprises benzylsulfonyl-D-Ser-homoPhe-(4-amidino-benzylamide), or a salt thereof, in a concentration ranging from 0.001 to 10,000 ppm related to the total weight of the cosmetic composition, and preferably in a concentration ranging from 0.1 to 1,000 ppm related to the total weight of the cosmetic composition. Most preferably, the cosmetic composition according to the present invention comprises benzylsulfonyl-D-Ser-homoPhe-(4-amidino-benzylamide), or a salt thereof, in a concentration ranging from 0.001 to 10,000 ppm related to the total weight of the cosmetic composition, and 0.1 to 15 wt-% glycerin calculated to the total weight of the cosmetic composition.

Preferred cosmetic compositions according to the present invention are cosmetic compositions further comprising one or several cosmetically acceptable carrier. The term cosmetically acceptable carrier refers to all carriers and/or excipients and/or diluents conventionally used in cosmetic compositions which are intended for topical use. In particular the cosmetic compositions are skin care preparations, hair care preparations, decorative preparations, and functional preparations such as particularly skin and hair care preparations.

Cosmetically acceptable carriers to be used in the present invention are in particular glycerin, polyglycerine compounds, ethylene glycol, propylene glycol, polyethylene glycols, polypropylene glycols, ethyl alcohol, isopropyl alcohol, agar gum, gum tragacanth, gum arabic, plant or animal gelatine, methyl cellulose, ethyl cellulose, carboxymethyl cellulose, hydroxymethyl cellulose, hydroxypropyl cellulose, sodium alginate, polyvinyl alcohol, polyvinyl alcohol acetate ester, C6-22 fatty alcohols such as cetyl alcohol, C6-22 fatty alcohol esters, in particular of stearic acid, palmitic acid, lauric acid and corresponding methyl, ethyl and propyl ester, lanolin, liquid paraffins or natural or synthetic waxes, such as vaseline or beeswax, vegetal oils such as olive oil, coconut oil, soybean oil, castor oil and corresponding hardened oils, hydroxyl-containing compounds modified with polyalkylene oxides, as well as further raw materials known to be incorporated in cosmetic formulations.

A cosmetic composition according to the present invention is a composition that can be in the form of a solution, an emulsion (oil/water or water/oil), a multiple emulsion (oil/water/oil, or water/oil/water), a microemulsion, a milk, a lotion, a dispersion, an ointment, a pomade, a shampoo, a soap, a gel, a lyophilisate, a powder, a stick, a pencil, a spray, a body oil, a face mask or a patch.

The present invention also provides the use of a cosmetic composition comprising benzylsulfonyl-D-Ser-homoPhe-(4-amidino-benzylamide), or a salt thereof, and a polyalcohol for maintaining, restoring, and/or improving the skin hydration, and/or the transepidermal water loss level in human skin.

The ability of the skin to hold water is primarily due to the stratum corneum and is critical for maintaining healthy and beautiful skin. Lipids arranged in an organized manner between the cells of the stratum corneum form a barrier to transepidermal water loss.

Transepidermal Water Loss (TWL) or (TEWL) is defined as the measurement of the quantity of water that passes from inside a body (animal or plant) through the epidermal layer (skin) to the surrounding atmosphere via diffusion and evaporation processes.

Measurements of TWL allow to identify skin damages caused by certain chemicals, or physical insult (such as “tape stripping”). However, TWL is also affected by environmental factors such as humidity, temperature, the time of year (season variation) and the moisture content of the skin (hydration level).

In another embodiment, the present invention also provides a cosmetic composition comprising benzylsulfonyl-D-Ser-homoPhe-O-amidino-benzylamide), or a salt thereof, and a polyalcohol for use in the treatment xerosis and/or dandruff by inhibition of plasmin enzymatic activity in the skin and/or scalp. Plasmin activity can easily be measured by the person skilled in the art using commercially available plasmin assay kits like for example SensoLyte® AFC Plasmin Assay Kit from Anaspec.

Moreover, the present invention also provides a cosmetic composition comprising benzylsulfonyl-D-Ser-homoPhe-(4-amidino-benzylamide), or a salt thereof, and a polyalcohol for use in the treatment xerosis and/or dandruff by the co-inhibition of both plasmin and urokinase enzymatic activity in the skin and/or scalp. Urokinase activity can easily be measured by the person skilled in the art using commercially available urokinase assay kits like for example SensoLyte® AFC Urokinase Activity Assay Kit from AnaSpec.

The composition according to the present application is best used in form of creams, lotions, gels, shampoos and the like, for the application to skin and/or scalp. The topically effective composition comprising benzylsulfonyl-D-Ser-homoPhe-(4-amidino-benzylamide), or a salt thereof, and a polyalcohol of the present invention can be made available or prepared in any application form desired. Thus, these formulations can be, e.g., an aqueous or anhydrous preparation, an emulsion or micro-emulsion of the water-in-oil (w/o) or oil-in-water (o/w) type, a multiple emulsion, e.g., of the water-in-oil-in-water (w/o/w) type, a gel, a shampoo, a solid, or an aerosol. The formulations of the present invention may be available as, e.g., a powder, a wet patch, a lotion, a cream or an ointment, shampoos and washing formulations, or in any other cosmetically approved form.

The cosmetic compositions of the present invention, can be used together with any further, usually applied and topically applicable skin care ingredient. Examples of additional skin care ingredients are derived from plants, algae, microalgae, yeasts, mushrooms, animals and microorganisms, synthetic and semi-synthetic substances, melatonin, urea, creatinin, dimethylethanolamine and derivatives thereof, amino acids and derivatives thereof (e.g. serine, glycine, asparagine, cysteine, glutamine, lysine, arginine, aspartic acid, glutamic acid. N-acetylcysteine, citrulline), proteins, their hydrolyzates and derivatives thereof (e.g. collagen, gelatins), albumin, casein, elastin, keratin, sericin, fibroin, fillagrin), growth factors and derivatives thereof (e.g. transforming growth factor, insulin-like growth factor, epidermal growth factor, acid and basic fibroblast growth factor, nerve growth factor, keratinocyte growth factor, hepatocyte growth factor, platelet derived growth factor, granulocyte-macrophage colony stimulating factor, vascular endothelial growth factor), enzymes and proteases and derivatives thereof (papain, bromelain, subtilisin, superoxide dismutases, lactoperoxidase, phospholipases, transglutaminases), enzyme inhibitors, protease inhibitors and derivatives thereof (e.g. tranexamic acid, soy bean trypsin inhibitor, Bowman Birk inhibitor, LEKTI, aprotinin, elafin, SLPI, α1-antitrypsin, α1-antichymotrypsin, cholesterol sulfate, leupeptin, chymostatin, tissue inhibitors of metalloproteases, Elhibin®, Colhibin®, compounds of the Pefabloc® series), co-enzymes and derivatives thereof (e.g. ubiquinon, nicotinamide, nicotinamide adenine dinucleotide, nicotinamide adenine dinucleotide phosphate, coenzyme A. coenzyme B12, flavin adenine dinucleotide, flavin mononucleotide), peptides such as di-, tri-, tetra-, penta- and hexapeptides and derivatives thereof (e.g. carnosine, H-PAla-Pro-Dab-NHBenzyl, Cu(II)-H-Gly-His-Lys-OH, H-Gly-Leu-Phe-OH, Elaidyl-Lys-Phe-Lys-OH, Palmitoyl-Lys-Val-Lys-OH, H-Lys-Pro-Val-OH, Palmitoyl-Lys-Val-Dab-OH, H-Arg-Ser-Arg-Lys-OH, Palmitoyl-Lys-Val-Dab-ThrOH, H-Gly-Pro-Arg-Pro-Ala-NH2, Palmitoyl-Lys-Thr-Thr-Lys-Ser-OH, Acetyl-Glu-Glu-Mot-Gln-Arg-Arg NW), carbohydrates such as mono-, di-, tri- and oligosaccharides and derivatives thereof (e.g. glucose, fructose, mannose, dihydroxyacetone, erythrulose, saccharose, trehalose, maltoses), polysaccharides and derivatives thereof (e.g. galactomannans, glucomannans, β-glucans, carrageenans, glycogen, chitosan, lentinans, lichenins, inulins, fucoses, alginates, xyloglucans, dextranes, amyloses, fructanes, xanthans, pullulan), glycosaminoglycans, their subunits and derivatives thereof (e.g. hyaluronan, chondroitin sulfates, heparin, glucuronic acid. Nacetylglucosamine), purins, pyrimidines, nucleotides, nucleosides and derivatives thereof (e.g. allantoin, uric acid, adenosine, adenosine monophosphate, adenosine 5′-triphosphate, kinetin), carboxylic acids and derivatives thereof (e.g. lactic acid, citric acid, glycolic acid, azelaic acid, salicylic acid, lipoic acid, pyrrolidon carboxylic acid, urocanic acids, caffeic acid), fatty acids and derivatives thereof (e.g. linoleic acid, oleic acid, palmitic acid, conjugated linoleic acid), lipids and derivatives thereof (e.g. squalane, squalene, monoglycerides, diglycerides, triglycerides, petrolatum, lanolin), sphingosines, sphingolipids, glycosphingolipids, sulfolipids and derivatives thereof (e.g. phytosphingosines, ceramides, glycoceramides, cerebrosides, gangliobrosides, sulfatides), phospholipids and derivatives thereof (phosphatidyl choline, phosphatidyl serine, phosphatidyl ethanolamine), sterols, phytosterols, saponins and derivatives thereof (e.g. cholesterol, sitosterol, stigmasterol, kampesterol, lupeol, glycyrrhizin), flavonoids and derivatives thereof (e.g. rutin, guercetin, genistein, fisetin, myricetin, luteolin, hesperetin, silybin, silymarin, apigenin), phenols, polyphenols and derivatives thereof (e.g. epigallocatechin, epigallocatechin gallate, resveratrol, nordihydroguaiaretic acid, ellagic acid, resorcinol), terpenes and derivatives thereof (e.g. glycyrrhetinic acid, farnesol, a-bisabolol, β-bisabolol), alkaloids and derivatives thereof (e.g. caffeine, theophylline, theobromine), benzofurans and derivatives thereof (e.g. usnic acid), trace elements (e.g. Zn, Se, Mn) and salts thereof, polyalcohols and derivatives thereof (e.g. glycerol, propylene glycol, butylene glycol, sorbitol, erythritol, hexanediols, phytantriol), antimicrobial ingredients, antimicrobial peptides and derivatives thereof (e.g. zinc pyrithione, defensins, cathelicidins, dermcidins, histatin). UV absorbers and derivatives thereof (e.g. benzoates, anthranilates, salicylates, cinnamates, benzophenones (such as Parsol™ 340), benzimidazoles, benzotriazoles (such as Tinosorb™ M), triazines (such as Tinosorb™ 5), polysilicones (such as Parsol™ SLX), titanium oxide, zinc oxide, melanin, avobenzone), vitamins, provitamins and derivatives thereof (e.g. vitamin A, vitamins of the B series, vitamin C. vitamin D, vitamin E), retinoids and derivatives thereof (e.g. retinol, retinal, tretinoin, isotretinoin, alitretinoin, etretinate, acitretin, tazarotene, bexarotene), carotenoids and derivatives thereof (e.g. α-carotene, β-carotene, lycopene, lutein, zeaxanthin, astaxanthin), chelating agents and derivatives thereof (e.g. EDTA, desferrioxamine, furildioxime), moisturizers (e.g. glycerol, butylene glycol, sorbitol, urea. N-acetylglucosamine, hyaluronic acid, glycosaminoglycans, amino acids, protein hydrolyzates, collagen, mono-, di, oligo- and polysaccharides, Pentavitin®, Phytaluronate®), agents regulating the epidermal barrier function (e.g. ceramides, cholesterol, fatty acids, squalane, phytosphingosine, lanolin, lecithin, petrolatum), skin-revitalizing and regenerating ingredients (e.g. Revitalin®-BT, yeast extracts, symphytum extract, ginkgo biloba extract), skin tightening and anti-wrinkle agents (e.g. centella asiatica, Vialox®, Syn®-ake, Pefa®-Tight, Matrixyl®, Biopeptide CL, Kollaren PP, elaidyl-Lys-Phe-Lys-OH, HArg-Ser-Arg-Lys-OH, Argireline, Collaxyl, Dermican LS 9745), soothing and anti-inflammatory agents (e.g. camomile extract, panthenol, niacinamide, zinc oxide, aloe Vera, calendula extract, licorice extract, hamamelis extract, Sensicalmine, Aiistine, H-Lys-Pro-Val-OH), anti-itching ingredients (e.g. Stimu-Tex®, evening primrose oil), anti-dandruff ingredients (e.g. allantoin, selenium sulfide, bifonazole, zinc pyrithione), desquamatory ingredients (e.g. alpha hydroxy acids, beta hydroxy acids), antioxidants (e.g. superoxide dismutase, ubiquinone, lipoic acid, vitamin E, green tea extract), sebum regulating and anti acne agents (e.g. Regu®-Seb, linoleic acid, pygeum africanum extract, thymus officinalis extract, resorcinol, salicylic acid), agents regulating stretch marks (e.g. gotu kola extract, Darutosid, Registril), agents regulating the skin immune system (e.g. arnica extract, Immucell®), skin lightening agents (e.g. α-arbutin, β-arbutin, kojic acid, magnesium ascorbyl phosphate, licorice extract. Melfade®, Melanostatine, acetyl-Asn-Ser-Leu-Asp-Phe-NH2), skin tanning agents (erythrulose, dihydroxyacteone, Melitane PP), anti-slimming agents (e.g. caffeine, theophylline, guarana extract, Regu®-Fade), agents regulating the cutaneous microcirculation (e.g. arginine, silybin, silymarin), agents regulating the primary features of rosacea such as flushing and nontransient erythema (e.g. metronidazoie, azelaic acid), agents regulating couperose and telangiectasia (e.g. silymarin), antifungal ingredients (e.g. ketokonazole, cyclopyrox, tea tree oil), and mixtures thereof.

In another embodiment, the present invention also provides a method for maintaining, restoring, and/or improving the skin hydration, and/or the transepidermal water loss level in human skin, said method comprising the steps of applying an effective amount of a composition comprising benzylsulfonyl-D-Ser-homoPhe-(4-amidino-benzylamide), or a salt thereof, and a polyalcohol to the skin and/or scalp of an individual, and observing the effect.

The present invention also relates to a method for the inhibition of plasmin enzymatic activity in the skin and/or scalp, said method comprising the use of benzylsulfonyl-D-Ser-homoPhe-(4-amidino-benzylamide), or a salt thereof, in combination with a polyalcohol, preferably glycerol.

The present invention further relates to a method for the co-inhibition of both plasmin and urokinase enzymatic activity in the skin and/or scalp, said method comprising the use of benzylsulfonyl-D-Ser-homoPhe-(4-amidino-benzylamide), or a salt thereof, in combination with a polyalcohol, preferably glycerol.

The present invention further relates to a method for synergistically improving the effect of benzylsulfonyl-D-Ser-homoPhe-(4-amidino-benzylamide), or a salt thereof, on the maintenance, restoration, and/or improvement of the skin hydration, and/or on the transepidermal water loss level in human skin, said method comprising the topical application of a cosmetic composition comprising benzylsulfonyl-D-Ser-homoPhe-(4-amidino-benzylamide), or a salt thereof, in combination with a polyalcohol, preferably glycerol on the skin of a subject in need thereof.

The invention is further illustrated by the following examples.

### EXAMPLES

### Example 1

### Preparation of an Emulsion

Ingredients of phase A are heated to 70° C. and ingredients of phase B to 75° C. Under stirring phase B is poored into phase A. The mixture is cooled to 50° C., homogenized and cooled to 30° C., Then ingredients of phase C and phase D are added. The emulsion is stirred until room temperature is reached.

| Phase | Ingredients | % wt/wt |
|---|---|---|
| A | Tego Care 450 | 3.00 |
| | Cetearyl alcohol | 2.25 |
| | Glyceryl stearate | 2.25 |
| | Cetiol 868 | 10.00 |
| | Squalane | 5.00 |
| B | Water | 66.99 |
| | Sodium hyaluronate | 5.00 |
| C | Glycerin | 5.00 |
| | Phenonip | 0.50 |
| D | Benzylsulfonyl-D-Ser-homoPhe- | 0.01 |
| | (4-amidino-benzylamide) | |

### Example 2

### Preparation of am Anti-Aging Cream

| Ingredients | % (w/w) |
|---|---|
| Glyceryl Myristate | 4.00 |
| Cetyl Alcohol | 2.00 |
| Steareth-2 | 2.00 |
| Steareth-21 | 2.00 |
| Isopropyl Myristate | 5.00 |
| Caprylic/Capric Triglyceride | 8.00 |
| BHT | 0.05 |
| Dimethicone | 2.00 |
| Preservative | qs |
| benzylsulfonyl-D-Ser-homoPhe- | 0.01 |
| (4-amidino-benzylamide) | |
| Water | Ad 100 |
| Xanthan Gum | 0.50 |
| Disodium EDETA | 0.10 |
| Glycerin | 5.00 |

### Example 3

### Cream for Baby Care and after Sun Cream

| Ingredients | INCI Name | wt.-% |
|---|---|---|
| Imwitor 372P | Glyceryl Stearate Citrate | 2.00 |
| Cutina GMS | Glyceryl Stearate | 3.00 |
| Sympatens-O/4200 | Sorbitan Laurate & Polyglyceryl-10 | 1.00 |
| Sweet Almond Oil | Prunus Amygdalus Culcis | 2.50 |
| Tegosoft TN | C12-15 Alkyl Benzoate | 7.00 |
| Cetiol OE | Dicaprylyl Ether | 5.00 |
| PENTAVITIN | Aqua, Saccharide Isomerate, Citric Acid | 5.00 |
| Tegosoft DC | Decyl Cocoate | 3.00 |
| BHT | BHT | 0.05 |
| Euxyl PE 9010 | Phenoxyethanol & Ethylhexylglycerin | 1.00 |
| Dow Corning 345 | Cyclopentasiloxane and | 2.00 |
| | Cyclohexasiloxane | |
| Keltrol RD | Xanthan Gum | 0.30 |
| Glycerin | Glycerin | 3.00 |
| Water | Aqua | ad 100 |
| Compound of | benzylsulfonyl-D-Ser-homoPhe- | 0.02 |
| present invention | (4-amidino-benzylamide) | |
| Rapithix A-60 | Sodium Polyacrylate and Hydrogenated | 0.30 |
| | Polydecene and Trideceth-6 | |
| | total | 100.00 |
| | Acid or Base if necessary to pH 5-6 | |

### Example 4

### Oil in Water Foundation for Sensitive Facial Skin

| Ingredients | INCI | wt.-% |
|---|---|---|
| Deionised Water | Aqua | ad 100 |
| Glycerin | Glycerin | 5.00 |
| Triethanolamine | Triethanolamine | 0.80 |
| Paratexin | Methylparaben EP | 0.20 |
| Keltrol | Xanthan Gum | 0.30 |
| Compound of | benzylsulfonyl-D-Ser-homoPhe- | 0.02 |
| present invention | (4-amidino-benzylamide) | |
| Titanium dioxide | C.I. 77891 | 4.57 |
| SunCROMA yellow | C.I. 77492 | 0.30 |
| iron oxide | | |
| SunCROMA red | C.I. 77491 | 0.13 |
| iron oxide | | |
| SunCROMA black | C.I. 77499 | 0.20 |
| iron oxide | | |
| DC 556 | Phenyl Trimethicone | 3.60 |
| Stearic Acid | Stearic Acid | 1.4 |
| Cetyl Alcohol | Cetyl Alcohol | 3.0 |
| Paratexin P | Propylparaben EP | 0.1 |

### Example 5

### Water in Oil Cream Extra Care Against Stressed Skin

| Ingredients | INCI | wt.-% |
|---|---|---|
| Cremophor WO-7 | PEG-7 Hydrogenated Castor | 2.50 |
| | Oil | |
| Elfacos ST-9 | PEG-45/Dodecyl Glycol | 2.00 |
| | Copolymer | |
| Cirebelle 303 | Synthetic Wax | 5.00 |
| Cirebelle 109L | Synthetic Wax | 7.20 |
| Miglyol 818 | Caprylic/Capric/Linoleic | 5.00 |
| | Triglyceride | |
| Eutanol G | Octyldodecanol | 7.50 |
| Cetiol OE | Dicaprylyl Ether | 9.20 |
| Deionised Water | Aqua | ad 100 |
| Glycerin | Glycerin | 7.00 |
| Propylene Glycol | Propylene Glycol | 2.00 |
| Euxyl PE9010 | Phenoxyethanol and | 0.80 |
| | Ethylhexylglycerin | |
| Compound of | benzylsulfonyl-D-Ser-homoPhe- | 0.05 |
| present invention | (4-amidino-benzylamide) | |

### Example 6

### Moisturizing Gel

| Ingredients | % (w/w) |
|---|---|
| Pemulen TR-1 (Acrylate/C10-30 | 0.80 |
| Alkyl Acrylate Crosspolymer) | |
| Biotin | 0.01 |
| EDETA BD (Disodium EDTA) | 0.10 |
| D-Panthenol | 0.20 |
| Hyasol BT (Sodium Hyaluronate) | 1.00 |
| Euxyl K 400 (Methyldibromo | 0.20 |
| Glutaronitrile & Phenoxyethanol) | |
| NaOH (30%) | 1.00 |
| Glycerin | 5.00 |
| Compound of present invention | 0.1 |
| (benzylsulfonyl-D-Ser-homoPhe- | |
| (4-amidino-benzylamide) | |
| Niacinamide | 0.50 |
| Citric Acid (10%) | q.s. |
| Water deionized | ad 100 |

### Example 7

### Inhibitory Effect of the Cosmetic Composition of the Present Invention in Inhibiting Urokinase

The measurements were carried out on a microplate reader at 25° C. The test medium consisted of 200 μl of Tris buffer (0.05 M; 0.154 M NaCl, 5% ethanol, pH 8.0), 25 μl of aqueous substrate solution (Bz-βAla-Gly-Arg-pNA, 0.18 mM and 0.09 mM), and 50 μl of human uPA solution (final concentration 150 U/ml). Two concentrations of the substrate and five concentrations of the inhibitor compositions consisting of 0.1% inhibitor and 99.9% of glycerine 86% were used. Three minutes after the addition of the enzyme, 25 μl of acetic acid (50%) was added to quench the reaction, and the optical density was measured at 405 nm.

A ki-value of 0.028 μM was calculated according to Dixon34 using a linear regression program.

### Example 8

### Inhibitory Effect of the Cosmetic Composition of the Present Invention Against Plasmin

The measurements were carried out on a microplate reader at 25′C. The test medium consisted of 200 μl of Tris buffer (0.05 M; 0.154 M NaCl, 5% ethanol, pH 8.0), 25 μl of aqueous substrate solution (Tos-Gly-Pro-Lys-pNA, 0.18 mM and 0.09 mM), and 50 μl of human plasmin solution (final concentration 0.06 CTA-U/ml). Two concentrations of the substrate and five concentrations of the inhibitor composition consisting of 0.1% benzylsulfonyl-D-Ser-homoPhe-(4-amidino-benzylamide) and 99.9% of glycerine 86% were used. Three minutes after the addition of the enzyme, 25 μl of acetic acid (50%) was added to quench the reaction, and the optical density was measured at 405 nm.

A Ki-value of 0.036 μM was calculated for benzylsulfonyl-D-Ser-homoPhe-(4-amidino-benzylamide according to Dixon34 using a linear regression proaram.

### Example 9

### Inhibitory Effect of the Other 3 4-Amidino Benzylamines Derivatives in the Same Assay Against Plasmin (Comparative Data)

The measurements were carried out on a microplate reader at 25° C. The test medium consisted of 200 μl of Tris buffer (0.05 M; 0.154 M NaCl, 5% ethanol, pH 8.0), 25 μl of aqueous substrate solution (0.18 mM and 0.09 mM), and 50 μl of human plasmin or human uPA solution (plasmin final concentration 0.06 CTA-U/ml, uPA final concentration 150 U/ml). Two concentrations of the substrates and five concentrations of the inhibitor compositions consisting of 0.1% inhibitor and 99.9% of glycerine 86% were used. Three minutes after the addition of the enzyme, 25 μl of acetic acid (50%) was added to quench the reaction, and the optical density was measured at 405 nm. The Ki-values for the respective inhibitors were calculated according to Dixon34 using a linear regression proaram. Bz-βAla-Gly-Arg-pNA was used as the substrate for human uPA and Tos-Gly-Pro-Lys-pNA for human plasmin.

| | | |
|---|---|---|
| | Ki for plasmin | Ki for uPA |
| Inhibitor composition | inhibition | inhibition |

| | | | | |
|---|---|---|---|---|
| Benzylsulfonyl-D-Ser-Gly- | 11 | μM | 0.036 | μM |
| (4-amidino-benzylamide) + | | | | |
| glycerin | | | | |
| Benzylsulfonyl-D-Ser-Ala- | 0.54 | μM | 0.0077 | μM |
| (4-amidino-benzylamide) + | | | | |
| glycerin | | | | |
| Benzylsulfonyl-D-Ser-Lys- | 0.36 | μM | 0.024 | μM |
| (4-amidino-benzylamide) + | | | | |
| glycerin | | | | |

This data shows Ki for plasmin inhibition is surprisingly an order of magnitude better for benzylsulfonyl-D-Ser-homoPhe-(4-amidino-benzylamide) compared to 3 other 4-amidino benzylamines as previously described in WO2009/026949.

### Example 10

### Selectivity of the Composition According to the Present Invention Against Other Serine Proteases

The measurements were carried out on a microplate reader at 37 CC. The test medium consisted of 140 μl of Tris buffer (0.05 M; 0.05% Brij® 35 P, pH 9.0), 20 μl of aqueous substrate solution (25 mM), and 20 μl of enzyme solution (final concentration 100 ng/ml). Three concentrations of the inhibitor composition consisting of 0.1% benzylsulfonyl-D-Ser-homoPhe-(4-amidino-benzylamide) and 99.9% of glycerine 86% were used (final concentrations: 100 ppm, 10 ppm and 1 ppm). The reaction kinetics were recorded during two hours at 405 nm. Boc-Phe-Ser-Arg-pNA was used as the substrate for human kallikrein 5, MeOSuc-Arg-Pro-Tyr-pNA for human kallikrein7, MeOSuc-Ala-Ala-Pro-Val-pNA for human elastase. Bz-βAla-Gly-Arg-pNA for human uPA and MeOSuc-Ala-Phe-Lys-pNA for human plasmin.

### Results:

| | Inhibition | | |
|---|---|---|---|
| | 100 ppm | 10 ppm | 1 ppm |
| Kallikrein 5 | 10% | 0% | 0% |
| Kallikrein 7 | 0% | 0% | 0% |
| Elastase | 7% | 0% | 0% |
| Urokinase | 100% | 88% | 41% |
| Plasmin | 100% | 94% | 64% |

1. Cosmetic composition comprising
a) benzylsulfonyl-D-Ser-homoPhe-(4-amidino-benzylamide), or a salt thereof, and
b) a polyalcohol.

2. A cosmetic composition according to claim 1 which is a topically applicable composition.

3. A cosmetic composition according to claim 1, wherein the polyalcohol is glycerol.

4. A cosmetic composition according to claim 1, wherein the benzylsulfonyl-D-Ser-homoPhe-(4-amidino-benzylamide), or a salt thereof, is used in a concentration ranging from 0.001 to 10,000 ppm related to the total weight of the cosmetic composition.

5. A cosmetic composition according to claim 1, wherein the benzylsulfonyl-D-Ser-homoPhe-(4-amidino-benzylamide), or a salt thereof, is used in a concentration ranging from 0.1 to 1,000 ppm related to the total weight of the cosmetic composition.

6. A cosmetic composition according to claim 1, further comprising one or several cosmetically acceptable carrier.

7. A cosmetic composition according to claim 1, wherein the composition can be in the form of a solution, an emulsion (oil/water, water/oil, water/oil/water, or oil/water/oil), a microemulsion, a milk, a lotion, a dispersion, an ointment, a pomade, a shampoo, a soap, a gel, a lyophilisate, a powder, a stick, a pencil, a spray, a body oil, a face mask or a patch.

8. Use of a cosmetic composition comprising
a) benzylsulfonyl-D-Ser-homoPhe-(4-amidino-benzylamide), or a salt thereof, and
b) a polyalcohol,
for maintaining, restoring, and/or improving the skin hydration, and/or the transepidermal water loss level in human skin.

9. A cosmetic composition comprising
a) benzylsulfonyl-D-Ser-homoPhe-(4-amidino-benzylamide), or a salt thereof, and
b) a polyalcohol
for use in the treatment xerosis and/or dandruff by inhibition of plasmin enzymatic activity in the skin and/or scalp.

10. A cosmetic composition comprising
a) benzylsulfonyl-D-Ser-homoPhe-(4-amidino-benzylamide), or a salt thereof, and
b) a polyalcohol
for use in the treatment xerosis and/or dandruff by the co-inhibition of both plasmin and urokinase enzymatic activity in the skin and/or scalp.

11. Method for maintaining, restoring, and/or improving the skin hydration, and/or the transepidermal water loss level in human skin comprising the steps of applying an effective amount of a composition according to claim 1 to the skin and/or scalp of an individual, and observing the effect.

12. Method for the inhibition of plasmin enzymatic activity in the skin and/or scalp, said method comprising the use of benzylsulfonyl-D-Ser-homoPhe-(4-amidino-benzylamide), or a salt thereof, in combination with a polyalcohol.

13. Method for the co-inhibition of both plasmin and urokinase enzymatic activity in the skin and/or scalp, said method comprising the use of benzylsulfonyl-D-Ser-homoPhe-(4-amidino-benzylamide), or a salt thereof, in combination with a polyalcohol.

14. Method for synergistically improving the effect of benzylsulfonyl-D-Ser-homoPhe-(4-amidino-benzylamide), or a salt thereof, on the maintenance, restoration, and/or improvement of the skin hydration, and/or on the transepidermal water loss level in human skin, said method comprising the topical application of a cosmetic composition according to claim 1 on the skin of a subject in need thereof.

15. Method according to claim 12, wherein the polyalcohol is glycerol.
